(19) United States
(12) Patent Application Publication
(10) Pub. No.: US 2015/0293106 A1
(43) Pub. Date: Oct. 15, 2015

(54) DIAROMATIC AMINO ACID SUBSTRATE FOR CATHEPSIN DETECTION

(71) Applicants: Dong Ig LEE, (US); INDUSTRY-ACADEMIC COOPERATION FOUNDATION, YONSEI UNIVERSITY, Seoul (KR)

(72) Inventors: Dong Ig Lee, Seoul (KR); Ik Sung Ahn, Seoul (KR); Chan Jin Kim, Seoul (KR); Chang Ha Lee, Seoul (KR)

(21) Appl. No.: 14/435,884

(22) PCT Filed: Feb. 13, 2013
(86) PCT No.: PCT/KR2013/001093

§ 371 Date: Apr. 15, 2015

(30) Foreign Application Priority Data
Oct. 16, 2012 (KR) 10-2012-0114666

(51) Int. Cl. G01N 33/574; G01N 33/58; C07K 5/065

(52) U.S. Cl. CPC G01N 33/57496; C07K 5/06078; G01N 33/581; G01N 2333/96466

(57) ABSTRACT

The present invention relates to a diaromatic amino acid substrate for cathepsin detection. When the substrate used for cathepsin detection is a polymer resulting from the binding of a protease-preferred amino acid and quencher to a diaromatic amino acid, cathepsin can be detected sensitively and accurately by fluorescence because cathepsin causes the protease-preferred amino acid and quencher to dissociate from the diaromatic amino acid, and fluorescence is recovered during this time.

### BACKGROUND

1. Field of the Invention

The present invention relates to the sensitive and accurate fluorescence detection of a cathepsin which is a cysteine protease, using diaromatic amino acid-based substrate.

2. Discussion of Related Art

Proteases are enzymes that selectively cleave the peptide bonds of proteins and polypeptides. Proteases are classified into a serine protease, a cysteine protease, an aspartic protease and a metalloprotease based on their mechanism of action. There have been increasing reports that proteases are related to a variety of diseases, for example, a viral infection such as HIV, cardiovascular diseases, cancers, Alzheimer's diseases and inflammatory diseases, and thereby the demand for—materials which can conduct a rapid, sensitive, and specific analysis of protease activity has been largely increased over the decades. For example, since cysteine proteases particularly play a role in the change of the extracellular matrix, antigen presentation, and processing events, cysteine proteases may represent various drug targets with respect to major diseases such as osteoporosis, arthritis, immune response-related diseases, arteriosclerosis, cancers, many parasitic infections, etc.

Among cysteine proteases, it has been known that cathepsins B, L and S are involved in tumor formation, growth, and invasion, and in various types of cancers, for example, high levels of cathepsin B are found in breast cancer, colon cancer, esophageal cancer, gastric cancer, lung cancer, ovarian cancer, and thyroid cancer as well as glioma and melanoma, high levels of cathepsin L are found in tumors of the kidney and testicles, non-small cell lung cancer, breast cancer, ovarian cancer, colon cancer, kidney cancer, bladder cancer, prostate cancer and thyroid cancer, and high levels of cathepsin S are found in high grade prostatic intraepithelial neoplasia and tumor-associated macrophages. Accordingly, these cathepsins have received much attention. In the case of squamous cell carcinoma of the skin, the expression of cathepsin L in keratinocytes shows that cathepsin L protects the cells from neoplastic progression. Such a protective role of cathepsin L suggests that there is a need for the development of substrates or inhibitors which can completely define the role of each cysteine cathepsin in malignant progression, and can identify cysteine cathepsins before therapeutic targeting. Only a small number of substrates and inhibitors have been developed for a specific cysteine cathepsin. That is, examples of these substrates and inhibitors are Abz-Gly-Ile-Val-Arg-Ala-Lys(Dnp)-OH for the selective analysis of cathepsin B, Abz-Leu-Glu-Gln-EDDnp for the selective analysis of cathepsin S, and AceHis-Gly-Pro-Arg-ACC which is a selective inhibitor for cathepsin K. Abz, Dnp, EDDnp, and ACC respectively refer to o-aminobenzoyl, 2,4-dinitrophenyl, ethylene diamine 2,4-dinitrophenyl, and 7-amino-4-carbamoylmethylcourmarin.

However, the above-described peptide-based substrates have a disadvantage of a high cost of peptide synthesis and a high manufacturing cost due to the complexity of equipment.

### SUMMARY OF THE INVENTION

The objective of the present invention is directed to providing a diaromatic amino acid-based substrate which may sensitively and selectively analyze a cathepsin, a method of preparing the same, and the use of the same.

In order to achieve the objective, one aspect of the present invention provides a compound of Formula 1:

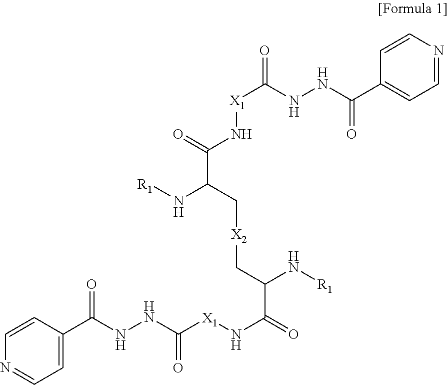

Where X₁ represents glycine, lysine, arginine or leucine,

X₂

represents

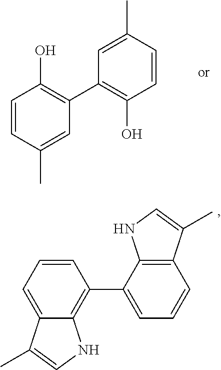

and

R₁ represents an amine-protecting group.

Further, another aspect of the present invention provides a method of preparing a compound of Formula 1 including

preparing a compound of Formula 4 by reacting a compound of Formula 2 with a compound of Formula 3;

removing an amine-protecting group R₁ from the compound of Formula 4; and

preparing the compound of Formula 1 by reacting a compound of Formula 4 from which the amine-protecting group R₁ is removed with a compound of Formula 5:

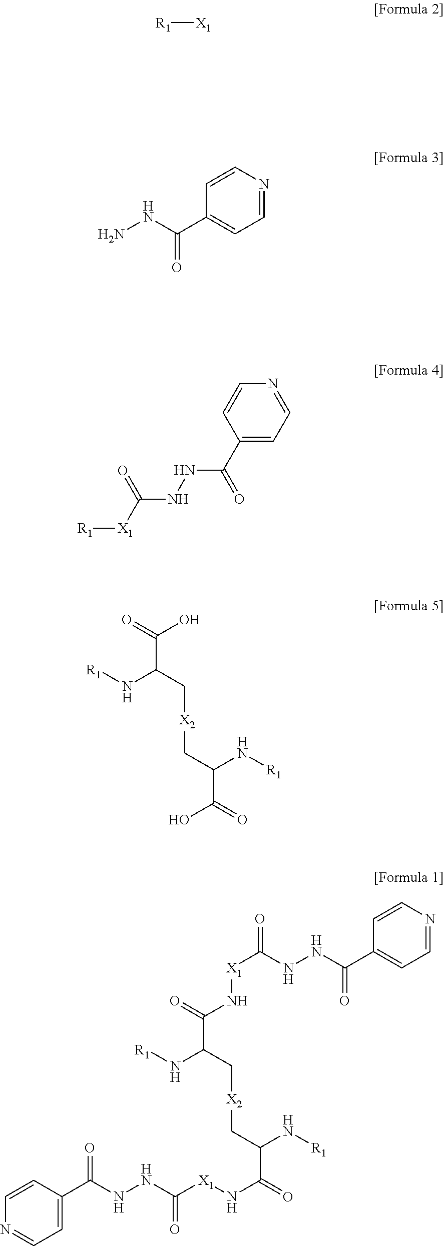

Where X₁ represents glycine, lysine, arginine or leucine,

X₂ represents

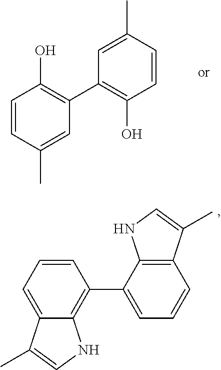

and

R₁ represents an amine-protecting group.

Further, still another aspect of the present invention provides a cathepsin-detecting sensor including a compound of Formula 1.

Further, yet another aspect of the present invention provides a cathepsin-detecting composition including a compound of Formula 1.

Further, yet another aspect of the present invention provides a diagnostic kit for a cathepsin-overexpressing disease including a compound of Formula 1.

Further, yet another aspect of the present invention provides a composition for screening cathepsin inhibitors including a compound of Formula 1.

### BRIEF DESCRIPTION OF THE DRAWINGS

The above and other objects, features, and advantages of the present invention will become more apparent to those of ordinary skill in the art by describing in detail exemplary embodiments thereof with reference to the attached drawings, in which:

FIG. 1 illustrates a synthesis process of DBDY-(amino acid-INH)₂.

FIG. 2 shows characterization results of separated and refined DBDY-(Gly-INH)₂, where FIG. 2(A) illustrates a chemical structure, FIG. 2(B) illustrates a HPLC chromatogram, and FIG. 2(C) illustrates a ¹H NMR spectrum in d6-DMSO.

FIGS. 3 to 10 show the chemical structures and ¹H NMR spectrums in d6-DMSO of separated and refined DBDY-(amino acid-INH)₂, in which, FIG. 3 shows DBDY-(Lys-INH)₂, FIG. 4 shows DBDY-(Ile-INH)₂, FIG. 5 shows DBDY-(Leu-INH)₂, FIG. 6 shows DBDY-(Met-INH)₂, FIG. 7 shows DBDY-(Phe-INH)₂, FIG. 8 shows DBDY-(Val-INH)₂, FIG. 9 shows DBDY-(Trp-INH)₂, and FIG. 10 shows DBDY-(Tyr-INH)₂.

FIGS. 11 to 19 show the hydrolysis results of 9 types of DBDY-(amino acid-INH)₂ catalyzed by a protease, FIG. 11 shows DBDY-(Gly-INH)₂, FIG. 12 shows DBDY-(Lys-INH)₂, FIG. 13 shows DBDY-(Ile-INH)₂, FIG. 14 shows DBDY-(Leu-INH)₂, FIG. 15 shows DBDY-(Met-INH)₂, FIG. 16 shows DBDY-(Phe-INH)₂, FIG. 17 shows DBDY-(Val-INH)₂, FIG. 18 shows DBDY-(Trp-INH)₂ and FIG. 19 shows DBDY-(Tyr-INH)₂.

FIG. 20 shows the hydrolysis results of DBDY-(Gly-INH)₂ (A) and DBDY-(Lys-INH)₂ (B) catalyzed by cysteine proteases including cathepsins B, L and S.

FIG. 21 shows a HPLC chromatogram of a hydrolysate catalyzed by cathepsin B of DBDY-(Gly-INH)₂ (A) and DBDY-(Lys-INH)₂ (B).

### DETAILED DESCRIPTION OF EXEMPLARY EMBODIMENTS

Hereinafter, exemplary embodiments of the present invention will be described in detail. However, the present invention is not limited to the embodiments disclosed below, but can be implemented in various forms. The following embodiments are described in order to enable those of ordinary skill in the art to embody and practice the present invention.

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

The terminology used herein is for the purpose of describing particular embodiments only and is not intended to be limiting of exemplary embodiments. The singular forms “a”, “an,” and “the” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, components, and/or groups thereof, but do not preclude the presence or addition of one or more other features, integers, steps, operations, elements, components, and/or groups thereof.

With reference to the appended drawings, exemplary embodiments of the present invention will be described in detail below. To aid in understanding the present invention, like numbers refer to like elements throughout the description of the figures, and the description of the same elements will be not reiterated.

Hereinafter, exemplary embodiments of the present invention will be described in detail.

The inventors of the present invention found out that DBDY-(INH)₂ may be synthesized by the binding of N,N′-diBoc-dityrosine (DBDY) and two molecules of isoniazid, and may be used in a selective and sensitive analysis of papain and chymopapain in advanced research. Regarding DBDY-(isoniazid)₂, DBDY and isoniazid were used as peptide components (DBDY=amino acid at a P₁ position, INH=amino acid at a P₂ position) specific to papain and chymopapain, and also as a fluorescence dye and a fluorescence quencher. However, DBDY-(INH)₂ was not hydrolyzed by cathepsins B and L. Further, it has been reported that most papain-like cysteine proteases including cathepsins B and L prefer arginine, lysine, leucine, and glycine at the P₁ position. Based on this report, the inventors of the present invention synthesized various types of DBDY-(amino acid-INH)₂ having amino acids identified by proteases, including glycine, lysine, or the like, at a P₁ position, thereby completing the present invention.

Accordingly, the present invention relates to a compound of Formula 1:

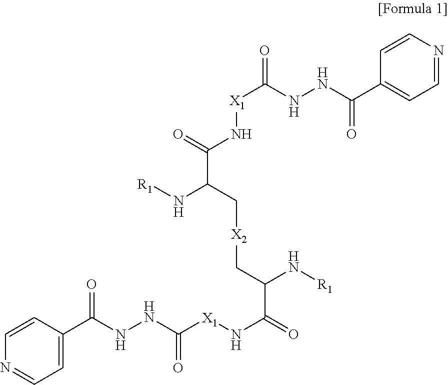

Where X₁ represents glycine, lysine, arginine or leucine,

X₂

represents

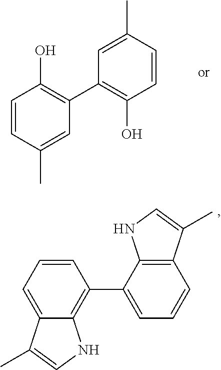

and

R₁ represents an amine-protecting group.

Terms used for the definition of the substitution product of the compound according to an embodiment of the present invention are below.

An “amine-protecting group” refers to one type of protecting group which may provide chemical selectivity for a specific functional group by binding to an amine group during a normal organic synthesis. The amine-protecting group refers to a well-known amine-protecting group, for example, such as a carbobenzyloxy group, a p-methoxybenzyl carbonyl, a tert-butyloxy carbonyl group, a 9-fluorenyl-methyloxy carbonyl group, an acetyl group, a benzyl group, a carbamate group, etc. Further, as the amine-protecting group, a hydrophilic or hydrophobic compound modified to a carboxyl group, or an amino acid may serve as a protecting group by binding to an amine group. Examples of the hydrophilic or hydrophobic compound may include polyethylene glycol, polypropylene glycol, polystyrene, polyisopyrene, polymethyl methacrylate, derivatives thereof, copolymers thereof, etc.

An “amino acid moiety” refers to a side chain bound to carbon of an amino acid, and may include a side chain portion except for an amino group and a carboxyl group of a well-known type of an amino acid, for example, an amino acid such as alanine, cysteine, aspartic acid, etc.

Further, in the present specification, regarding the term “protease-preferred amino acid”, an N terminal side of a substrate is named P₁, P₂, . . . Pₙ, and a C terminal side of a substrate is named P₁′, P₂′, . . . P′ₙ according to the nomenclature of Schechter & Berger, and a hydrophobic amino acid such as Phe, Tyr or the like is preferred at a P₂ position, and an amino acid such as Arg, Lys, Gly, Leu, or the like is preferred at a P₁ position in most of the papain-like cysteine proteases. Accordingly, according to an embodiment of the present invention, a diaromatic amino acid which maintains hydrophobicity is positioned at a P₂ position, an amino acid such as Gly, Lys, or the like is positioned at a P₁ position, and thereby the selective degradation by a papain-like cysteine protease may be induced. Further, the quencher may be bound to a P₁′ position. Accordingly, a protease-preferred amino acid refers to an amino acid present at sites such as P₁, P₂, or the like of a substrate as described above.

In the compound of Formula 1, specifically, X₁ represents glycine or lysine,

X₂ represents

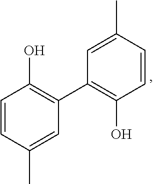 [SMILES: Cc1ccc(O)c(-c2cc(C)ccc2O)c1]

R₁ is a tert-butyloxycarbonyl group, a 9-fluorenylmethyloxycarbonyl group, a benzyloxycarbonyl group, polyethylene glycol, or NH₂CHR′ₙ, where R′ is an amino acid moiety, and n represents an integer in the range of 1 to 20.

The compound of Formula 1 according to the embodiment of the present invention is a composite polymer which may be used as a substrate capable of specifically detecting a protease, particularly a cathepsin, and which is prepared by binding a protease-preferred amino acid and a quencher compound which removes the fluorescence of a diaromatic amino acid to the both terminals of a diaromatic amino acid through a direct bond or a crosslinking bond with a carboxyl group which is a fluorescent functional group terminal of a diaromatic amino acid.

Regarding the compound according to the embodiment of the present invention, the diaromatic amino acid has a structure in which an amine-protecting group is bound to an amine group terminal, or an amine group is blocked by a hydrophilic or hydrophobic compound, or an amino acid bound to the amine group through an amide bond for the chemical selectivity of a carboxyl group terminal.

Further, the amine group terminal of the quencher compound of Formula 1 bonds to the carboxyl group of a protease-preferred amino acid through a reaction, the amine group of the protease-preferred amino acid reacts with the carboxyl group of a diaromatic amino acid to form a peptide bond, the protease-preferred amino acid and the quencher compound are bound to the both terminals of the diaromatic amino acid, and thus the compound according to the embodiment of the present invention may be prepared.

The fluorescence of the diaromatic amino acid is removed by the peptide bond between the diaromatic amino acid and a protease-preferred amino acid-quencher compound. The peptide bond site of the diaromatic amino acid and the quencher compound is a recognition site which may be degraded by a protease, particularly, a cathepsin, and the quencher compound is removed from a diaromatic amino acid-based substrate through the degradation by a cathepsin, and thus the fluorescence of the diaromatic amino acid is restored. Accordingly, since the compound according to the embodiment of the present invention shows the increase of the fluorescence intensity through the degradation depending on the presence of a cathepsin, and thus the fluorescence detection of a cathepsin may be achieved, the compound according to the embodiment of the present invention may be used as a fluorescent sensor for detecting a cathepsin.

Further, the present invention relates to a method of preparing the compound of Formula 1, including

preparing a compound of Formula 4 by reacting a compound of Formula 2 with a compound of Formula 3;

removing an amine-protecting group R₁ from the compound of Formula 4; and

preparing the compound of Formula 1 by reacting a compound of Formula 4 from which the amine-protecting group R₁ is removed with a compound of Formula 5:

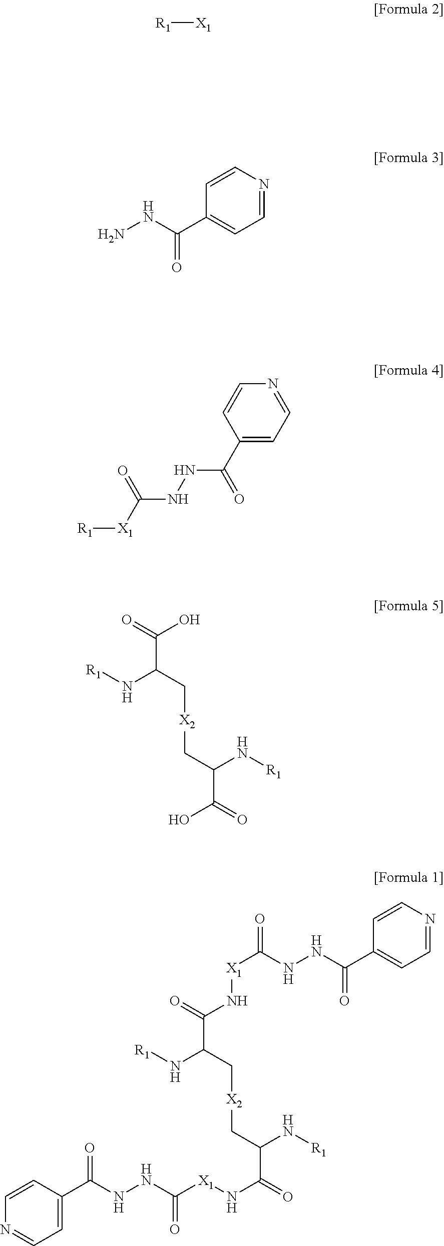

Where X₁ represents glycine, lysine, arginine or leucine,

X₂ represents

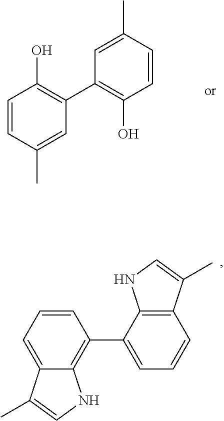

and

R₁ represents an amine-protecting group.

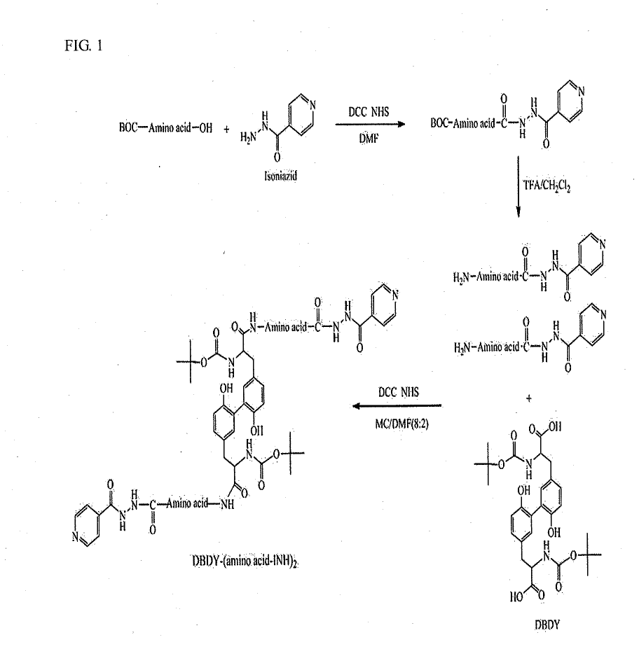

Referring to FIG. 1, a method of preparing the compound of Formula 1 according to the embodiment of the present invention is described in detail as below.

The first step is a step of preparing the compound of Formula 4 by reacting the carboxyl group of the amino acid of Formula 2 having an amine-protecting group bonded with one terminal thereof with the amine group of the quencher of Formula 3 in the presence of a catalyst and a crosslinking agent.

A well-known amine-protecting group may be used as the amine-protecting group without limitation, and a well-known method may be used as the introduction method of the amine-protecting group as well. An example of the amine-protecting group include a tert-butyloxycarbonyl group, a 9-fluorenylmethyloxycarbonyl group, a benzyloxycarbonyl group, polyethylene glycol, or NH₂CHR′ₙ, where R′ represents an amino acid moiety, and n may be an integer in the range of 1 to 20.

As the amino acid of Formula 2, the type of the amino acid preferred by protease may be used, and an example thereof includes glycine, lysine, arginine or leucine. In the case of the lysine, lysine has a side chain of (—(CH₂)₃CH₂NH₂), and thus may be used in the form in which FMOC is further bound thereto upon the binding of the lysine and a diaromatic amino acid, so as to prevent the side chain from binding to the diaromatic amino acid. The FMOC may be removed by dissolving in a DMF solution containing 20% piperidine after the synthesis of the compound of Formula 1.

Regarding the quencher compound of Formula 3, any quencher compound including a functional group which may bind to the amino acid of Formula 2, or of which the terminals may be modified and activated to include the functional group may be used without limitation, but the quencher compound preferably may be isoniazid (INH).

The binding reaction between the amino acid of Formula 2 and a quencher compound may be carried out under the presence of a catalyst and a crosslinking agent.

The catalyst and crosslinking agent may be used to activate the terminal functional groups of the amino acid of Formula 2 or the quencher compound of Formula 3 to be in the form of an acyl halide (Cl and F), an acyl azide, an activated ester, an acyl isourea(carbodiimide) anhydride, an acyloxyphosphonium cation, and an acyluronium cation.

An example of the catalyst may include a carbodiimide-based compound such as 1-ethyl-3-(3-dimethylaminopropyl)carbodiimide hydrochloride (EDC), 1-cyclohexyl-3-(2-morpholinoethyl)carbodiimide (CMC), N,N′-dicyclohexyl carbodiimide (DCC), diisopropyl carbodiimide) (DIC), or the like, and NHS or sulfo-NHS; N,N′-carbonyldiimidazole; or Woodward's reagent K, etc.

An example of the crosslinking agent may include a homobifunctional crosslinking agent such as dithiobis(succinimidylpropionate) (DSP), 3,3′-dithiobis(sulfosuccinimidylpropionate) (DTSSP), disuccinimidyl suberate (DSS), bis(sulfosuccinimidyl)suberate (BS3), ethylene glycolbis(succinimidylsuccinate) (EGS), N,N′-disuccinimidyl carbonate (DSC), or the like, or a heterobifunctional crosslinking agent such as N-succinimidyl 3-(2-pyridyldithio)propionate (SPDP), succinimidyloxycarbonyl-α-(2-pyridyldithio)toluene (SMPT), succinimidyl 4-(N-maleimidomethyl)-cyclohexane-1-carboxylate (SMCC), etc.

According to an embodiment, an amide bond may be formed by changing the amine group of the compound of Formula 3 to an activated ester using a carbodiimide-based compound and NHS, and then reacting the activated ester with the carboxyl group of the compound of Formula 2.

Further, the reaction may be carried out with stirring at room temperature in a solvent such as dichloromethane, dimethylformamide, 1,4-dioxane, methylene chloride, etc.

More specifically, dimethylformamide may be used as the solvent. This is because a carboxyl group activated by NHS has high reactivity with an amine group in dimethylformamide.

The compound of Formula 4 bonded with a protease-preferred amino acid and a quencher compound may be prepared using 1 to 3 moles of the compound of Formula 3 per mole of the compound of Formula 2.

The compound of Formula 4 may be, for example, glycine-INH, lysine-INH, arginine-INH or leucine-INH, having a protecting group bonded with one terminal thereof.

In a method of preparing the compound of Formula 1 according to the embodiment of the present invention, the second step is the step of removing an amine-protecting group from the compound of Formula 4, thereby ensuring a functional group which may react with the diaromatic amino acid of Formula 5.

The amine-protecting group may be removed by adding the compound of Formula 4 into a mixed solvent in which trifluoroacetic acid (TFA) and methylene chloride (CH₂Cl₂) are mixed at the same amounts and stirring the mixed solvent, but the present invention is not particularly limited thereto.

In a method of preparing the compound of Formula 1 according to the embodiment of the present invention, the third step is the step of preparing the compound of Formula 1 by reacting the compound of Formula 4 of which one terminal has a functional group, particularly, an amine group from which the amine-protecting group was removed in the above-described step, with the carboxyl group of the diaromatic amino acid of Formula 5.

The compound of Formula 1 may be prepared through the reaction between the carboxyl group of the diaromatic amino acid of Formula 5 of which the amine groups at both terminals are blocked and the amine group of the amino acid-isoniazid compound of the Formula 4.

The diaromatic amino acid of Formula 5 may be dityrosine or ditryptophan.

As the diaromatic amino acid of the Formula 5, it is preferable to use a diaromatic amino acid of which the amine groups at both terminals are protected or blocked for the optimum reaction of the terminal carboxyl group.

The block of the diaromatic amino acid may be performed by introducing an amine-protecting group, or treating with a compound which may form an amide bond with the amine group of the diaromatic amino acid.

An example of the protecting group includes a well-known amine-protecting group, and is not particularly limited. Further, a well-known method may also be performed as the introduction method thereof.

An example of the compound which may form an amide bond with the amine group of the diaromatic amino acid may include a hydrophilic or hydrophobic compound modified to a carboxyl group, for example, such as polyethylene glycol, an amino acid, or the like, but is not particularly limited thereto.

The binding reaction between the diaromatic amino acid and the protease-preferred amino acid-quencher compound may be carried out in the presence of a catalyst and a crosslinking agent.

The catalyst and crosslinking agent may be used to activate the terminal functional groups of the diaromatic amino acid or the amino acid-quencher compound of Formula 4 to be in the form of an acyl halide (Cl and F), an acyl azide, an activated ester, an acyl isourea(carbodiimide) anhydride, an acyloxyphosphonium cation, and an acyluronium cation.

The types of the catalyst and crosslinking agent are the same as described above.

According to an embodiment, an amide bond may be formed by changing the amine group of the compound of Formula 4 to an activated ester using a carbodiimide-based compound and NHS, and then reacting the activated ester with the carboxyl group of the diaromatic amino acid.

According to another embodiment, an urea bond may be formed by changing the amine group of the compound of Formula 4 to an activated ester using DSC which is one of the homofunctional crosslinking agents, and then reacting the activated ester with the amine group of the diaromatic amino acid.

Further, the reaction may be carried out with stirring the compounds at normal temperature in a solvent of one or a mixture of two types of dichloromethane, dimethylformamide, 1,4-dioxane, methylene chloride, etc. More specifically, a mixture of methylene chloride and dimethylformamide at a volume ratio of 8:2 may be used as the solvent. This is because a carboxyl group activated by NHS has high reactivity with an amine group in dimethylformamide.

As a result of the reaction, a polymer in which protease-preferred amino acid-quencher compounds are bound to the both terminals of the diaromatic amino acid may be obtained in 24 hours with high yield.

2 to 5 moles of the compound of Formula 4 may be used per mole of the compound of Formula 5. More specifically, about 3 moles of the compound of Formula 4 may be used per mole of the compound of Formula 5. When the compounds are used in the above-described range, protease-preferred amino acid-quenchers may be bound to the both terminals of the diaromatic amino acid.

Further, the method of preparing the compound according to the embodiment of the present invention further includes the step of separating and refining the compound prepared after the reaction through a precipitation method and dialysis using a solvent, silica columns, gel permeate chromatography (GPC), gel filtration chromatography (GFC), or ion exchange chromatography (IEC).

Further, the present invention relates to a cathepsin-detecting sensor including a compound of Formula 1.

Since the peptide bond of the compound of Formula 1 according to an embodiment of the present invention is degraded by a cathepsin, the fluorescence of a diaromatic amino acid is restored, the intensity of fluorescence is increased, and thus the compound of Formula 1 according to the embodiment of the present invention may be used as a fluorescent sensor to selectively detect a cathepsin.

As shown in FIG. 1, fluorescence signaling shows the turn-on type fluorescence signaling characteristics, in which the peptide bond between a protease-preferred amino acid and a quencher compound is degraded from a diaromatic amino acid-based substrate, the quencher compound is separated from the compound of Formula 1, and thus the fluorescence of the diaromatic amino acid is restored, showing fluorescence with high intensity.

Since the compound of Formula 1 according to the embodiment of the present invention shows the selective increase of fluorescence at wavelengths of 284 nm (excitation wavelength) and 410 nm (emission wavelength) in a concentration-dependent manner as a cathepsin is added to an aqueous solution, the compound of Formula 1 may be used as a “turn-on” type fluorescent probe to detect a cathepsin.

The cathepsin-detecting sensor according to the embodiment of the present invention may be provided as a general kit which may determine the presence and concentration of a cathepsin by mixing a cathepsin with a sample solution to detect a cathepsin therefrom.

The cathepsin may be cathepsin B or cathepsin L.

Further, the present invention relates to a cathepsin-detecting composition including a compound of Formula 1.

Further, another aspect of the present invention relates to a method of detecting a cathepsin, including reacting the compound of Formula 1 with a sample to detect a cathepsin therefrom; and measuring the intensity of fluorescence generated from the sample.

The compound of Formula 1 according to an embodiment of the present invention is a “turn-on” type sensor which may detect a cathepsin in an aqueous solution state, and is in a type of sensor of which the fluorescence intensity increases when the sensor with no fluorescence senses a cathepsin.

A cathepsin-detecting composition according to an embodiment of the present invention may include a buffer solution in addition to the compound of Formula 1. The type and concentration of the buffer solution is not particularly limited, and may be suitably modified according to the use of the cathepsin-detecting composition. Specifically, the buffer solution may have a pH in the range of 3 to 10. More specifically, a phosphate buffer solution including NaCl and EDTA and having the pH of 3 to 10 may be used, but the present invention is not particularly limited thereto.

Further, the detection of a cathepsin by the compound of Formula 1 denotes the measurement of the change of the fluorescence intensity, the compound of Formula 1 shows the highly-increased fluorescence intensity at a wavelength of 410 nm in a cathepsin concentration-dependent manner, and thus the fluorescence intensity may be measured.

Further, the present invention relates to a diagnostic kit for a cathepsin-overexpressing disease including a compound of Formula 1.

In the case of cathepsins, cathepsin B is overexpressed in breast cancer, colon cancer, esophageal cancer, gastric cancer, lung cancer, ovarian cancer, and thyroid cancer as well as glioma and melanoma, cathepsin L is overexpressed in tumors of the kidney and testicle, non-small cell lung cancer, breast cancer, ovarian cancer, colon cancer, kidney cancer, bladder cancer, prostate cancer and thyroid cancer, and thus the compound of Formula 1 may diagnose a disease overexpressing the cathepsin enzymes through the fluorescence detection of a cathepsin.

The diagnosis of a cathepsin-overexpressing disease may be performed by comparing the fluorescence intensity of a cathepsin in a normal cell with the fluorescence intensity of a cathepsin in a diseased cell.

Further, the present invention relates to a composition for screening for cathepsin inhibitors including a compound of Formula 1.

Regarding the screening composition according to the embodiment of the present invention, a cathepsin and a sample including a candidate substance as a cathepsin inhibitor are reacted using the compound of Formula 1, the cathepsin inhibitor suppresses the activity of the cathepsin enzyme, the degradation of the peptide bond of the compound of Formula 1 accordingly is suppressed, and thus a decrease of the fluorescence intensity may be measured, and the screening composition according to the embodiment of the present invention can screen the candidate substance as a cathepsin inhibitor.

The composition according to an embodiment of the present invention may include the composition of the compound of Formula 1 and control reagents (positive and/or negative) formulated in the dosage form. For example, the composition may be orally administered as a sugar-coated tablet, a capsule, an elixir and a microcapsule according to the need, or may be parenterally administered in the form of an injection of a sterile solution having water or any pharmaceutically acceptable liquid or a suspension.

Further, the compound of Formula 1 may be mixed with a pharmaceutically acceptable carrier or medium, such as sterilized water, physiological saline, a vegetable oil, an emulsifier, a suspending agent, a surfactant, a stabilizer, a fragrant agent, an excipient, a vehicle, a preservative, a binder, etc. The amount of active ingredient may be adjusted as a suitable dose in the suitable range.

An example of an additive which may be mixed in a tablet and capsule includes a binder such as gelatin, corn starch, tragacanth gum, and arabic gum; an excipient such as a crystal cellulose; a swelling agent such as corn starch, gelatin, and alginic acid; a lubricant such as magnesium stearate; a sweetening agent such as sugar, lactose or saccharin; and a fragrant agent such as peppermint, gaultheria adenothrix oil and cherry.

When the unit dosage form is a capsule, a liquid carrier such as an oil may be further included in the components. A sterile composite for injection may be formulated according to a normal drug recipe using vehicle such as sterilized water used in an injection. Physiological saline and another isotonic solution including an adjuvant such as glucose, D-sorbitol, D-mannitol, D-mannose, and sodium chloride may be used as an aqueous solution of an injection. This may be used with a suitable solvent such as an alcohol, more specifically, a polyalcohol such as ethanol, propylene glycol and polyethylene glycol, and a non-ionic surfactant such as polysorbate 80™ and HCO-50.

Further, the composition according to the embodiment of the present invention may be produced in a kit form, and thus include instruction kits (e.g., paper, tape, VCR, CD-ROM, etc.) to perform analysis. The analysis format of the composition is a relative fluorescence value using a fluorescence spectrometer well known in the related field, for example, such as Perkin-Elmer LS55 luminescence spectrometer.

Hereinafter, the present invention will be described in detail by means of Examples. However, it should be understood that the following Example are given by way of illustration of the present invention only, and are not intended to limit the scope of the present invention.

### Example 1

### Synthesis of Dityrosine-Based Substrate for Detecting Cathepsin

Boc-tyrosine-OH (Boc-Tyr) (98%), Boc-phenylalanine-OH (Boc-Phe) (≧99%), Boc-tryptophan-OH (Boc-Trp) (≧99%), Boc-glycine-OH (Boc-Gly) (≧99%), Boc-valine-OH (Boc-Val) (≧99%), Boc-leucine-OH (Boc-Leu) (≧99%), Boc-tyrosine-OSu (Boc-Tyr-OSu) (≧99%), Boc-phenylalanine-OSu (Boc-Phe-OSu) (≧98%), Boc-tryptophan-OSu (Boc-Trp-OSu), INH, N,N-dicyclohexylcarbodiimide (DCC) (99%), N,N′-diisopropylcarbodiimide (DIC) (99%), N-hydroxysuccinimide (NHS), L-cysteine, ethylenediaminetetraacetic acid (EDTA), Tris-HCl, sodium phosphate monobasic, sodium phosphate dibasic, trifluoroacetic acid (TFA) (99%), N,N′-dimethylformamide DMF) (99.8%), deuterium oxide (D2O), dimethylsulfoxide-D6 ([D6]-DMSO) (99.9%), and dimethylsulfoxide were all purchased from Sigma-Aldrich Chemical Co. (St Louis, Mo., USA). Boc-lysine(FMOC)-OH (Boc-Lys(FMOC)-OH) (≧98%), Boc-isoleucine-OH.0.5H₂O (Boc-Ile.0.5H₂O) (≧98%), and Boc-methionine-OH (Boc-Met) (≧98%) were purchased from Novabiochem (Darmstadt, Germany). Acetonitrile and water (HPLC grade) were purchased from Baker Chemical Co. (Phillipsburg, N.J., USA). Silica 60 aluminum sheets were purchased from Merck Co. (Darmstadt, Germany). MgSO₄ was purchased from Showa Chemical Industry Co. (Meguro-ku, Tokyo, Japan). A PBS buffer solution was purchased from Hyclone Laboratories, Inc. (South Logan, Utah, USA). Sodium chloride, calcium chloride, and methylene chloride (99%) were purchased from Duksan Pure Chemical Co. (Ansan, GyonggiDo, Korea). For filtering, an Advantec PTFE filter membrane (Advantec, Japan) having a pore size of 0.2 μm was used. All proteases used in the embodiments of the present invention were purchased from Sigma-Aldrich Chemical Co.: chymotrypsin and trypsin derived from fetal bovine pancreas subtilisin from bacillus licheniformis, proteinase K derived from tritirachium album, thermolysin derived from bacillus thermoproteolyticus rokko, carboxy peptidase A derived from fetal bovine pancreas, dispase derived from bacillus polymyxa, collagenase derived from clostridium histolyticum, pepsin derived from pig gastric mucosa, aspergillopepsin derived from aspergillus saitoi, papain and chymopapain derived from papaya latex, bromelain derived from pineapple stem, cathepsins B and L derived from a human liver, and cathepsin S derived from a human spleen.

### (Synthesis of DBDY-(Amino Acid-INH)₂)

FIG. 1 schematically illustrates the synthesis of DBDY-(amino acid-INH)₂, which is formed by the binding of a Boc-amino acid and INH, the removal of Boc, and the binding of DBDY. According to the embodiment of the present invention, 9 types of DBDY-(amino acid-INH)₂ were synthesized: DBDY-(Tyr-INH)₂; DBDY-(Phe-INH)₂; DBDY-(Trp-INH)₂; DBDY-(Val-INH)₂; DBDY-(Gly-INH)₂; DBDY-(Ile-INH)₂; DBDY-(Leu-INH)₂; DBDY-(Met-INH)₂; and DBDY-(Lys-INH)₂.

Boc-Tyr-INH, Boc-Phe-INH, Boc-Trp-INH, and Boc-Val-INH were synthesized by mixing a Boc-amino acid (i.e., Boc-Tyr, Boc-Phe, Boc-Trp, and Boc-Val), INH, and DIC in a molar ratio of 2:1:2 in DMF. The mixture was reacted at room temperature overnight, and then freeze-dried to remove DMF. The remaining residue was cleaned using methylene chloride (CH₂Cl₂), and removed. Boc-Gly-INH, Boc-Ile-INH, Boc-Leu-INH, Boc-Met-INH, and Boc-Lys(FMOC)-INH were synthesized by mixing a Boc-amino acid, INH, NHS, and DCC in a molar ratio of 1:2:1:1 in DMF. The mixture was reacted at room temperature overnight, N,N′-dicyclohexylurea, which was a precipitate, was filtered for removal, and a filtrate was freeze-dried. The remaining residue was dissolved in CH₂Cl₂, cleaned three times using water, dried using anhydrous MgSO₄, and then vacuum-evaporated. The product was separated and refined in a preparative HPLC system mounted with a UV absorbance detector (Waters 486 Tunable Absorbance Detector, Meadows Instrumentation Inc., USA). The separation process was performed in a C-18 column (particle size: 5 μm, 250 mm×4.6 mm, pore size: 80 Å, Phenomenex, USA) using a mixture of water containing 0.1%-TFA and acetonitrile as an eluent. The elution was performed for 30 minutes, while the linear gradient of acetonitrile was increased from 0 to 40%. The flow rate and separation temperature of the eluent were maintained at 1 mL min⁻¹ and 35° C. The synthesized Boc-amino acid-INH was dissolved in TFA/CH₂Cl₂ (1:1), stirred at room temperature for 30 minutes, and thereby a Boc functional group was removed. After the evaporation of a solvent, the product was separated and refined in a C-18 column (particle size: 10 μm, 100 mm×21.2 mm, pore size: 110 Å, Phenomenex) in a preparative HPLC system using a mixture of water containing 0.1%-TFA and acetonitrile as an eluent. The elution was performed for 15 minutes, while the linear gradient of acetonitrile was increased from 0 to 40%. The flow rate and separation temperature of the eluent were maintained at 5 mL min^{d} and 35° C. The product was detected at wavelengths of 210 nm and 280 nm. Fractions containing a pure amino acid-INH were put into the product, and freeze-dried.

The synthesis method of DBDY is specifically disclosed in “Large-scale production of N,N-diBoc-dityrosine and dityrosine by HRP-catalyzed N-Boc-L-tyrosine oxidation and one-step chromatographic purification” written by D. I. Lee et. Al (Process Biochem. 46 (2011) 142-147).

DBDY, an amino acid-INH, NHS, and DCC were mixed in a molar ratio of 1:3:2.1:2.1 in CH₂Cl₂/DMF (8:2) for the binding of DBDY and the amino acid-INH. TEA was added into the solution at the same number of moles as the amino acid-INH in advance. The mixture was reacted at room temperature overnight, N,N′-dichlorohexylurea, which was a precipitate, was filtered and removed, and the filtrate was freeze-dried. The remaining reactant was cleaned using water and removed. The precipitate insoluble in water was separated and refined using a preparative HPLC system.

In the case of lysine having a side chain of (—(CH₂)₃CH₂NH₂), Boc-Lys(FMOC)-OH was used to prevent the side chain from binding to DBDY. After the separation and refinement of DBDY-(Lys(FMOC)-INH)₂, DBDY-(Lys(FMOC)-INH)₂ was dissolved in a DMF solution containing 20% piperidine, FMOC was removed. The mixture was stirred at room temperature for 30 minutes, and then freeze-dried. After the mixture was recrystallized in DMF-ethyl ether, a precipitate was filtered, collected, and then separated and refined using a preparative HPLC system.

The structural identification of DBDY-(amino acid-INH)₂ was performed based on a ¹H-NMR spectrum recorded at a Bruker Advance 500-MHz nuclear magnetic resonance spectrometer (Bruker BioSpin Corp., Germany).

### (Synthesis of Boc-Aromatic Amino Acid-Gly-INH and Boc-Aromatic Amino Acid-Lys-INH)

In order to evaluate the effect of DBDY in the selective analysis of cysteine proteases, a substrate without DBDY (i.e., Boc-amino acid-INH) and a substrate having an aromatic amino acid at a P₂ position (i.e., Boc-aromatic amino acid-amino acid-INH) were synthesized, and then compared with DBDY-(amino acid-INH)₂.

To this end, Boc-aromatic amino acid-Gly-INH and Boc-aromatic amino acid-Lys-INH were prepared by binding a Boc-aromatic amino acid with Gly-INH and Lys(FMOC)-INH. A Boc-aromatic amino acid-OSu solution included in DMF and a Gly-INH or Lys(FMOC)-INH solution included in DMF were mixed in a molar ratio of 1:1. TEA was added into the mixture at the same number of moles as the Gly-INH and Lys(FMOC)-INH solution in advance. After the mixture was reacted at room temperature overnight, the residue was freeze-dried. The remaining reactant was cleaned using water and CH₂Cl₂, and removed. In the case of Boc-aromatic amino acid-Lys(FMOC)-INH, a FMOC functional group was removed. The product (i.e., Boc-aromatic amino acid-Gly-INH and Boc-aromatic amino acid-Lys-INH) was separated and refined using a preparative HPLC system, and the structure of the product was identified using a ¹H-NMR spectrum.

### (Quenching Efficiency of Isoniazid and Amino Acid)

In order to compare the quenching efficiencies of INH and amino acid in DBDY-(amino acid-INH)₂, the fluorescence of DBDY was measured according to the presence of INH and amino acid. In order to remove the reabsorption and reemission effects, the concentration of DBDY was adjusted to 0.25 μM, the concentrations of INH and amino acid were adjusted to 10 mM except for tyrosine in a PBS buffer solution (pH 7.4, 25° C.). The concentration of tyrosine was adjusted to 5 mM due to low solubility in a PBS buffer solution having the pH of 7.4. The fluorescence intensity was measured using a PerkinElmer LS 55 luminescence spectrometer. The excitation wavelength (λₑₓ) and the emission wavelength (λₑₘ) were respectively adjusted to 320 nm and 410 nm.

### (Hydrolysis Catalyzed by Protease)

For the meaningful comparison of proteases, a hydrolysis reaction was performed under the optimum conditions as reported in other documents (regarding chymotrypsin, subtilisin, thermolysin and pepsin, refer to R. Beynon, J. S. Bond, Proteolytic Enzymes (Second Edition), Oxford University Press, 2001, pp. 295-316; regarding trypsin, proteinase K, carboxy peptidase A, collagenase, dispase, aspergillopepsin, papain, chymopapain, bromelain, cathepsin B, cathepsin L, and cathepsin S, refer to the catalog of Sigma-Aldrich Corporation).

Table 1 shows a summary of reaction conditions for the 16 types of proteases.

TABLE 1
Conditions for hydrolysis of DBDY-(amino
acid-INH)₂ catalyzed by protease

| Protease | Condition |
|---|---|
| Chymotrypsin | 50 mM phosphate buffer solution (pH |
| | 7.8, 25° C.) + 100 mM NaCl |
| Trypsin | 50 mM Tris-HCl buffer solution |
| | (pH 7.5, 25° C.) |
| Subtilisin | 50 mM Tris-HCl buffer solution (pH 7.5, |
| | 37° C.) + 10 mM CaCl₂ |
| Thermolysin | 50 mM Tris-HCl buffer solution (pH 7.5, |
| | 37° C.) + 10 mM CaCl₂ and 2.0M NaCl |
| Carboxy peptidase A | 50 mM Tris-HCl buffer solution (pH 7.5, |
| | 25° C.) + 500 mM NaCl |
| Collagenase | 50 mM Tris-HCl buffer solution (pH 7.5, |
| | 25° C.) + 10 mM CaCl₂ and 400 mM |
| | NaCl |
| Dispase & proteinase K | 50 mM Tris-HCl buffer solution (pH 7.5, |
| | 37° C.) + 2 mM CaCl₂ |
| Pepsin | 10 mM HCl solution (pH 2.0, 37° C.) |
| Aspergillopepsin | 0.2M glycine-HCl (pH 2.8, 37° C.) |
| Papain & Chymopapain | 50 mM-phosphate buffer solution (pH |
| | 6.2, 25° C.) + 5 mM L-cysteine, 300 mM |
| | NaCl, 2.0 mM EDTA |
| Bromelain | 0.1M-phosphate buffer solution (pH 6.5, |
| | 37° C.) + 5 mM L-cysteine, 300 mM |
| | KCl, 0.1 mM EDTA |
| Cathepsin B & Cathepsin L | 50 mM-phosphate buffer solution (pH |
| | 6.0, 25° C.) + 5 mM L-cysteine, 200 mM |
| | NaCl, 2 mM EDTA |
| Cathepsin S | 50 mM-phosphate buffer solution (pH |
| | 6.5, 25° C.) + 5 mM L-cysteine, 200 mM |
| | NaCl, 2 mM EDTA |

Before the reaction, the mixtures of the reaction buffer solutions and proteases were previously incubated for 30 minutes in a water bath in which the temperature was adjusted to an analysis temperature. The hydrolysis reaction was initiated by adding a substrate dissolved in DMSO. The volume of the added substrate solution was controlled such that the final concentration of DMSO was lower than 1.5% (v/v).

When DBDY-(amino acid-INH)₂ was hydrolyzed, the restored fluorescence of DBDY was measured using a PerkinElmer LS 55 luminescence spectrometer. The λₑₓ and λₑₘ were respectively adjusted to 284 nm and 410 nm with respect to papain, chymopapain, bromelain, cathepsins, aspergillopepsin and pepsin, and the λₑₓ and λₑₘ were respectively adjusted to 320 nm and 410 nm with respect to chymotrypsin, trypsin, subtilisin, proteinase K, thermolysin, carboxypeptidase A, collagenase and dispase. The reaction was performed under alkaline conditions.

Just after DBDY-(amino acid-INH)₂ was added, the fluorescence was measured, and expressed as an initial fluorescence. The fluorescence measured at the end of the reaction was expressed as a final fluorescence. The increased fluorescence was defined as the difference between the initial fluorescence and the final fluorescence, which is the same as the difference in fluorescence between the generated DBDY (or DBDY-(amino acid)₂) and the reacted DBDY-(amino acid-INH)₂.

A hydrolysate was measured using a HPLC system. In order to prepare a sample, a hydrolysis reaction was terminated by adding 2% (v/v) 1M HCl with respect to a serine protease, cysteine protease and metalloprotease, and adding 1M NaOH with respect to an aspartic protease. The separation was performed using the mixture of 10 mM-ammonium bicarbonate and acetonitrile as an eluent in C-18 column (particle size: 5 μm, 250 mm×4.6 mm, pore size: 80 Å, Phenomenex, USA). The elution was performed for 15 minutes, while the linear gradient of acetonitrile was increased from 0 to 60%. The flow rate and separation temperature of the eluent were maintained at 1 mL min⁻¹ and 35° C. The hydrolysate was detected at a wavelength of 254 nm.

### Experimental Example 1

### Synthesis and Characterization of DBDY-(Amino Acid-INH)₂

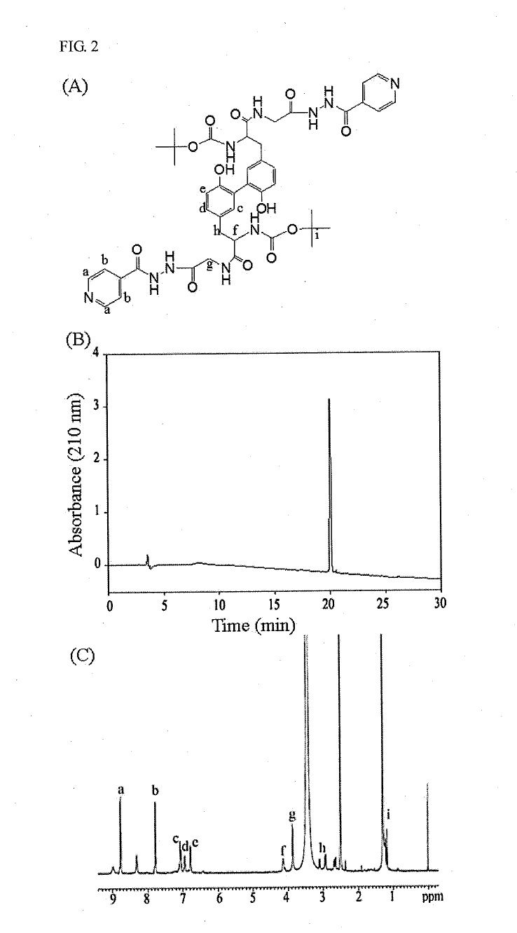

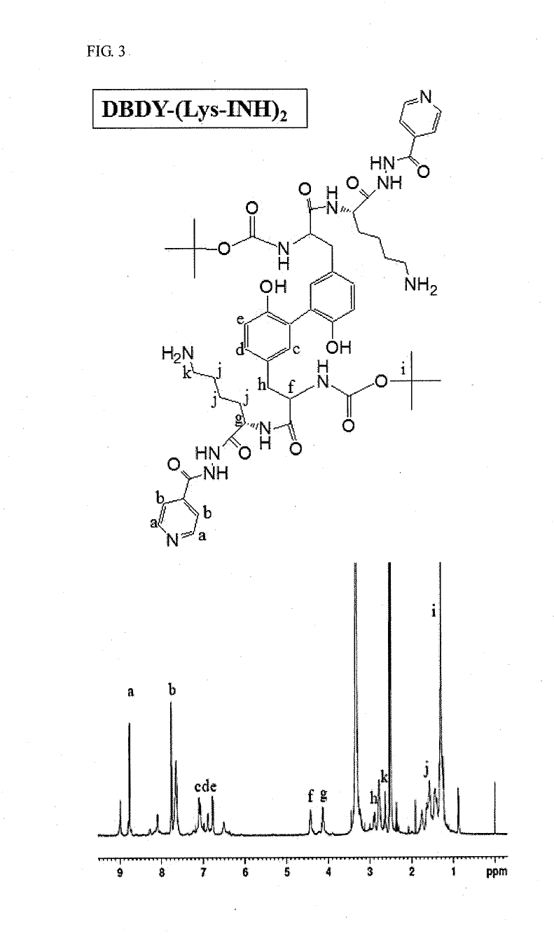

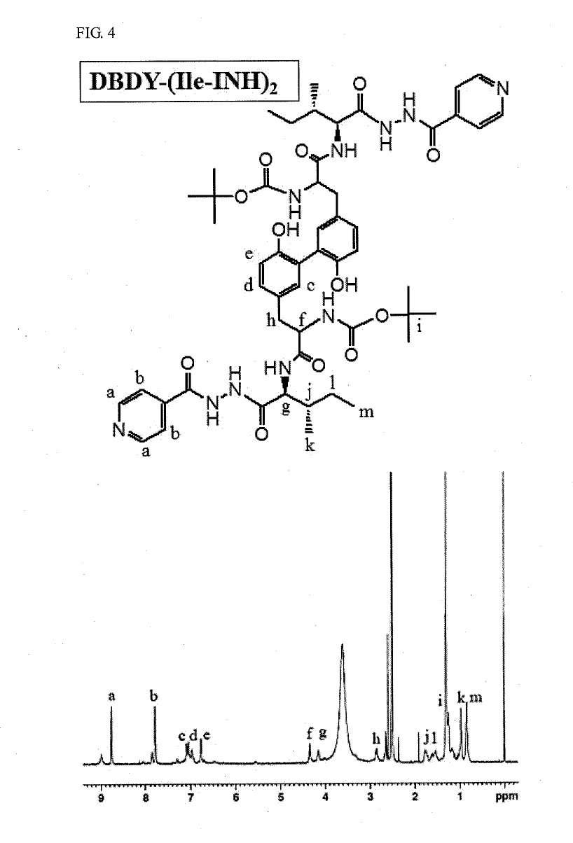

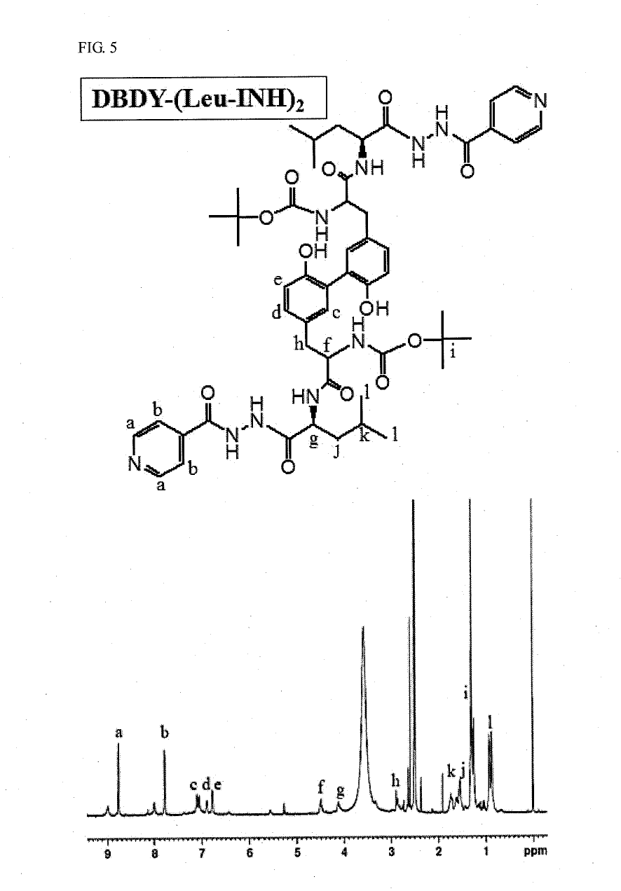

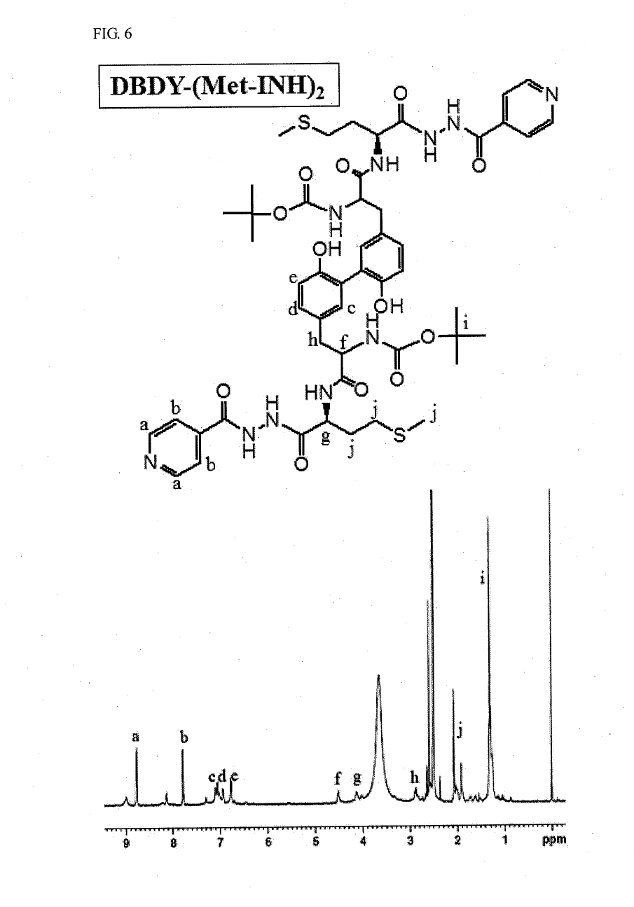

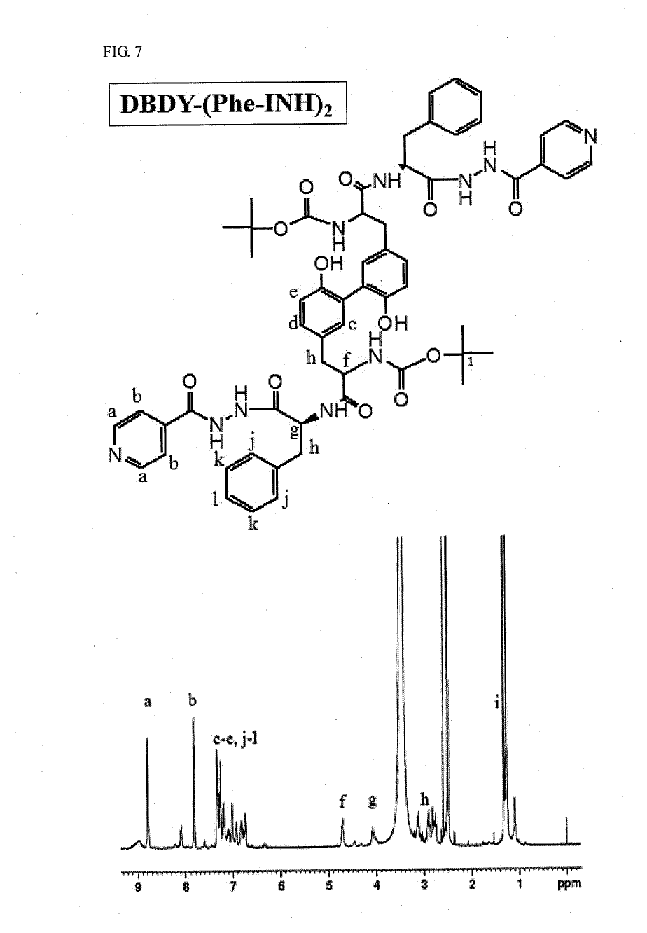

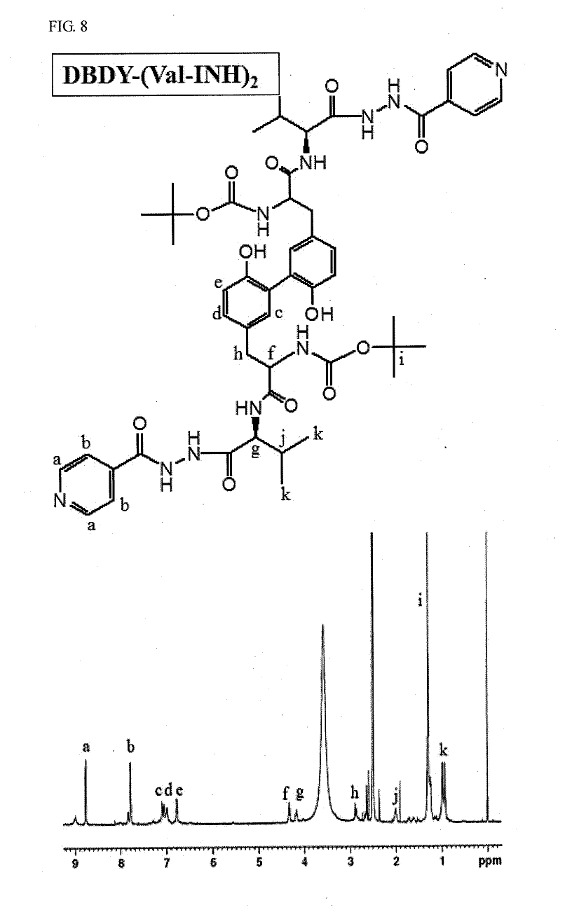

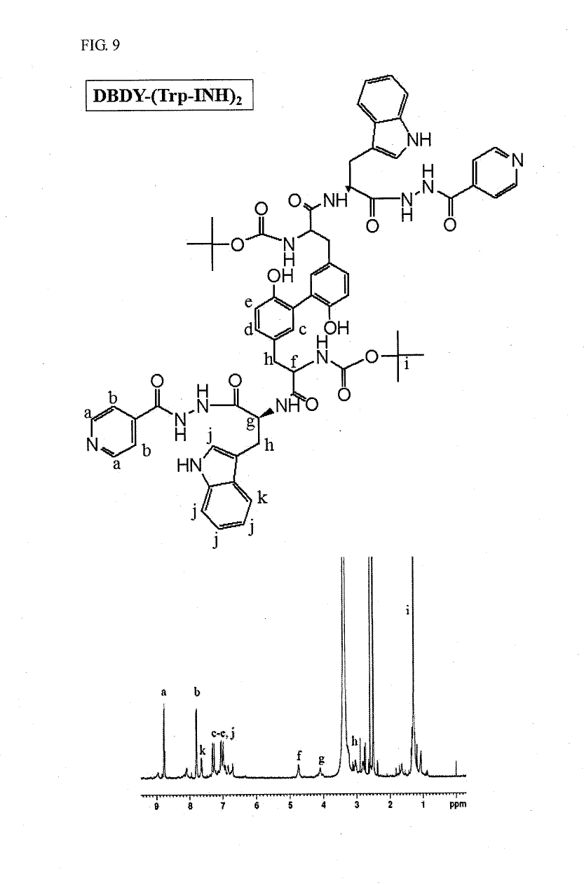

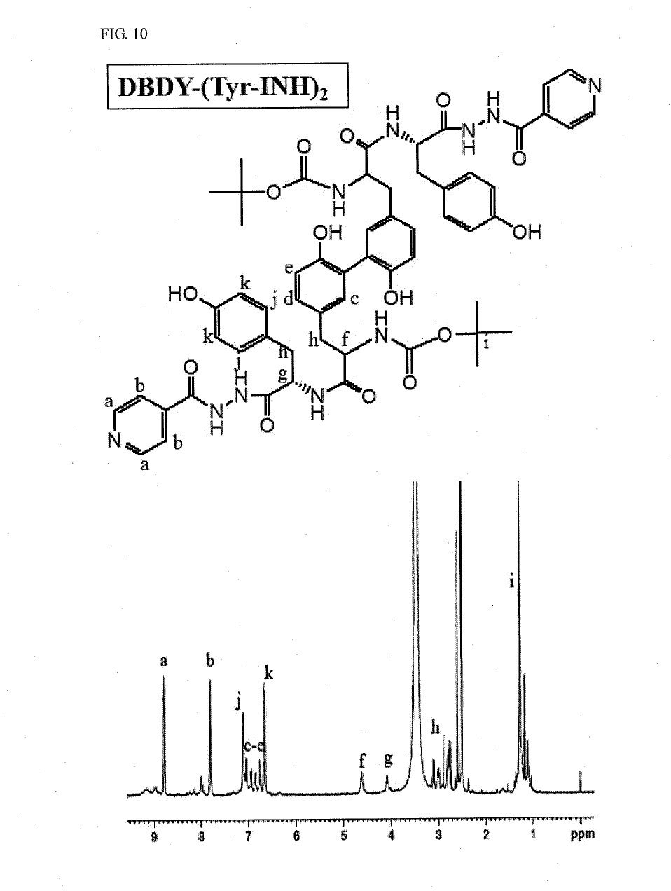

The purity and chemical structure of the synthesized DBDY-(amino acid-INH)₂ were measured from a HPLC chromatogram and ¹H NMR spectrum (Bruker Advance 500-MHz nuclear magnetic resonance spectrometer (Bruker BioSpin Corp., Germany)). FIG. 2 shows a result for DBDY-(Gly-INH)₂, where the HPLC chromatogram shows the presence of DBDY-(Gly-INH)₂ (RT=20.1 min) and the absences of DBDY (RT=28.0 min) and Gly-INH (RT=2.9 min), and RT refers to a retention time. The formula structures and ¹H NMR spectrum results in d6-DMSO of DBDY-(amino acid-INH)₂ per type were represented in FIGS. 2 to 10.

The fluorescence intensity of DBDY was measured in the presence of INH or an amino acid in DBDY-(amino acid-INH)₂, and the ratio between the fluorescence intensity of DBDY as measured above and the fluorescence intensity of DBDY itself was defined as a relative fluorescence quantum yield (refer to Table 2).

TABLE 2
Relative fluorescence quantum yield of DBDY, Φ_{f,rel}(DBDY, i)

| Type | Φ_{f,rel}(DBDY, i) ^{a} |
|---|---|
| None | 1.00 |
| Lysine | 1.03 |
| Glycine | 0.96 |
| Valine | 0.99 |
| Phenylalanine | 0.93 |
| Tyrosine (5 Mm) ^{b} | 1.43 |
| Tryptophan | 2.74 |
| Leucine | 0.96 |
| Isoleucine | 0.96 |
| Methionine | 0.90 |
| Isoniazid | 0.25 |

${ }^{a} {Φ}_{f , rel} ( DBDY , i ) = \frac{fluorescence intensity of DBDY in the presence of i}{fluorescence intensity of DBDY only}$ Fluorescence was measured in a PBS buffer solution (pH 7.4, 25° C.) including 0.25 M-DBDY and 10 mM-amino acid or INH (except for tyrosine) (λₑₓ = 320 nm and λₑₘ = 410 nm).
^{b} Tyrosine was used at a concentration of 5 mM due to low solubility in a PBS buffer solution.

Only INH showed the meaningful removal of the fluorescence of DBDY in the presence of tyrosine and tryptophan (i.e., Φ_{f,rel} (DBDY, Tyr) and Φ_{f,rel} (DBDY, Trp)), and the values were higher than 1.0. This is because the fluorescence intensities of 5 mM-tyrosine and 10 mM-tryptophan were added to that of 0.25 μM-DBDY. When the fluorescence intensities were measured at the same concentrations (λₑₓ=320 nm and λₑₘ=410 nm), the fluorescence intensity of DBDY was about 11,000 times that of tyrosine and was about 4,300 times that of tryptophan. Accordingly, INH is the dominant influence on the removal of fluorescence in DBDY-(amino acid-INH)₂, and the fluorescence of DBDY was shown only when a hydrolysate was DBDY-(amino acid)₂.

### Experimental Example 2

### Evaluation of Selectivity of DBDY-(Amino Acid-INH)₂ for Protease Analysis

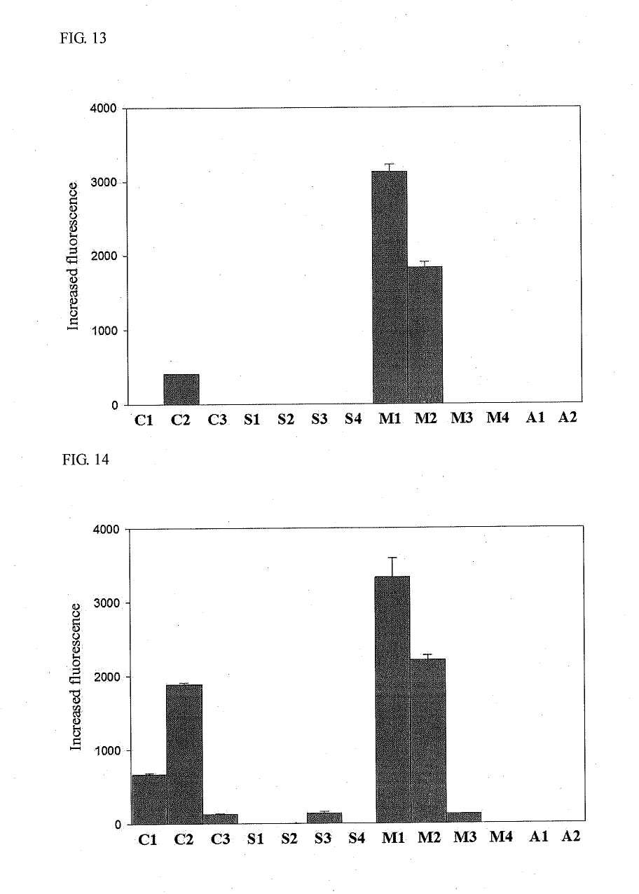

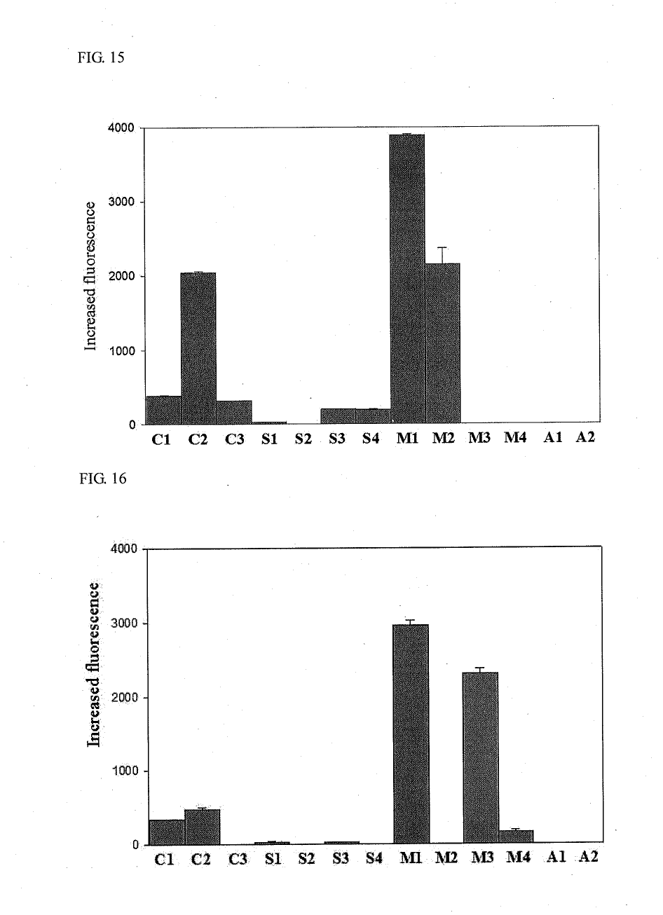

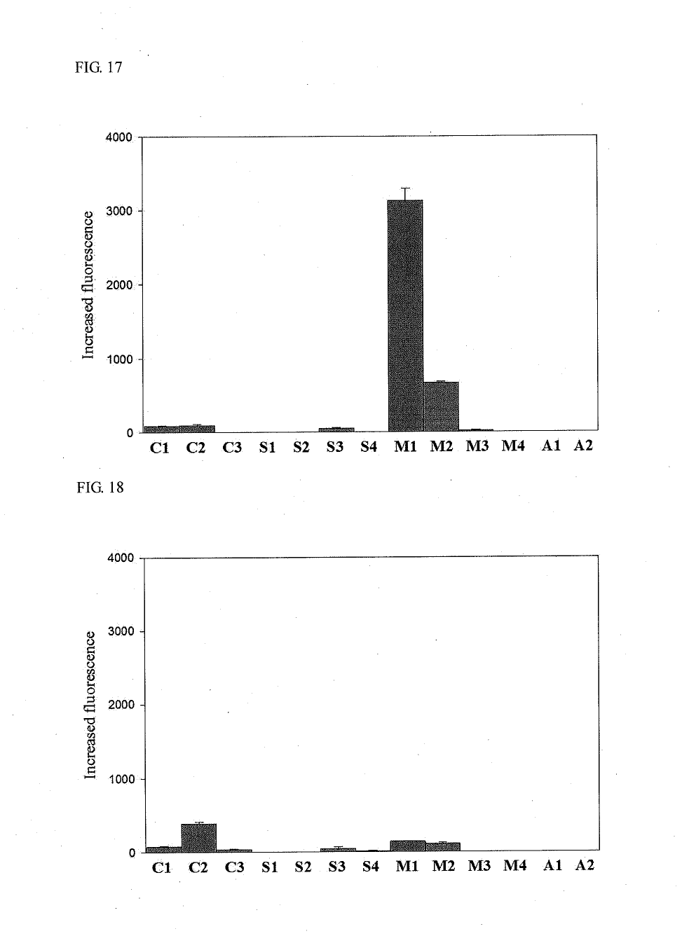

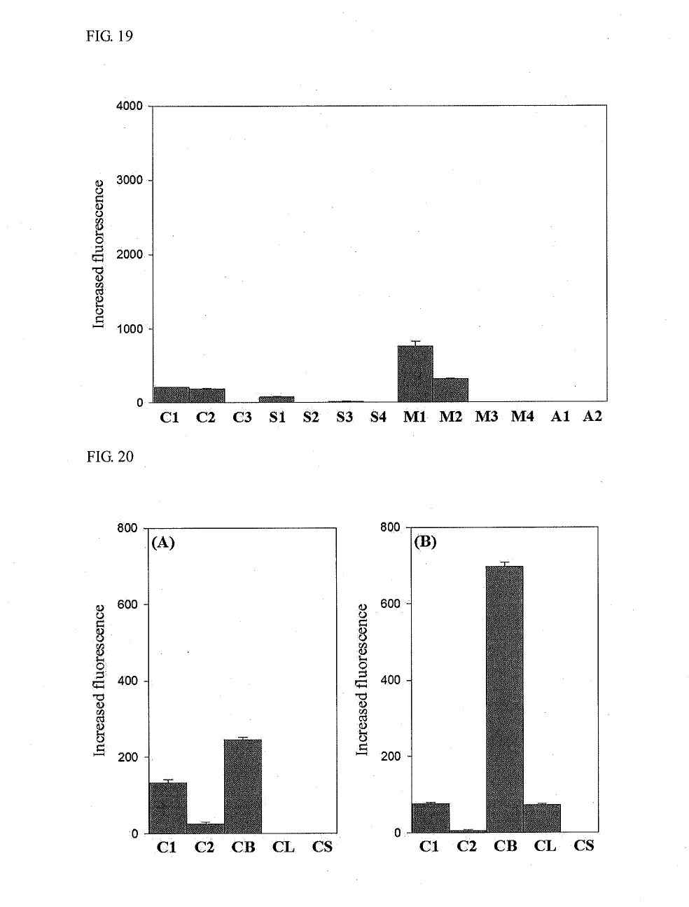

For the selective analysis of 13 types of proteases without cathepsin, the experiments on 9 types of DBDY-(amino acid-INH)₂ were performed, and the experiment results were illustrated in FIGS. 11 to 19. The initial concentrations of each substrate and protease were respectively set to 2.5 μM and 0.1 μM. The hydrolysis reaction was performed for two hours. Reference numbers on an X-axis denoted the following: C1=papain; C2=chymopapain; C3=bromelain; S1=chymotrypsin; S2=trypsin; S3=subtilisin; S4=proteinase K; M1=thermolysin; M2=carboxypeptidase A; M3=dispase; M4=collagenase; A1=pepsin; and A2=aspergillopepsin. Each experiment was repeated at least three times.

Among the substrates in the experiments, no substrate was hydrolyzed by the aspartic protease (i.e., pepsin and aspergillopepsin) and carboxypeptidase A. As shown in FIG. 11, DBDY-(Gly-INH)₂ showed sensitivity and selectivity for the cysteine protease (i.e., no hydrolysis by other proteases), and this seems to result from the interactive effects of DBDY (P₂ position) and Gly (P₁ position).

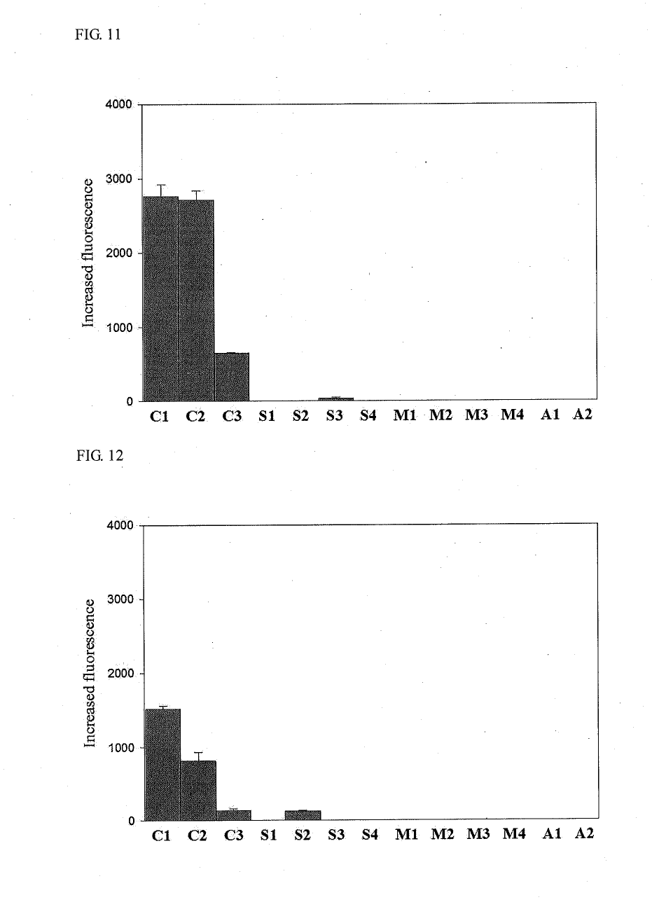

In corresponding to a report that most papain-like cysteine proteases and trypsin prefer lysine at a P₁ position, DBDY-(Lys-INH)₂ was hydrolyzed by trypsin as well as by cysteine proteases (refer to FIG. 12). There has been a report that thermolysin and dispase prefer peptides having Phe, Ile, Leu, Met, and Val at a P₁′ position. In corresponding to the above-described report, DBDY-(Phe-INH)₂, DBDY-(Ile-INH)₂, DBDY-(Leu-INH)₂, DBDY-(Met-INH)₂, and DBDY-(Val-INH)₂ showed high sensitivity for thermolysin and dispase (FIGS. 12 to 17), and the hydrolysates were Phe-INH, Ile-INH, Leu-INH, Met-INH, and Val-INH (not shown).

Although. DBDY-(Lys-INH)₂ was slightly hydrolyzed by trypsin, it could be clarified that DBDY-(Gly-INH)₂ and DBDY-(Lys-INH)₂ have the selectivity for cysteine proteases.

### Experimental Example 3

### Effect of Dityrosine with Respect to Selective Hydrolysis of DBDY-(Gly-INH)₂ and DBDY-(Lys-INH)₂ by Cysteine Proteases

In order to investigate the effect of dityrosine upon selective hydrolysis by cysteine proteases, DBDY was substituted with Boc-amino acid in DBDY-(Gly-INH)₂ and DBDY-(Lys-INH)₂. Based on the report that a hydrophobic amino acid such as phenylalanine was preferred at a P₂ position in most of the papain-like cysteine proteases, Boc-aromatic amino acid-Gly-INH and Boc-aromatic amino acid-Lys-INH were prepared in particular, and experimented as a substrate with respect to chymotrypsin, trypsin, papain, pepsin and thermolysis. The test results were summarized in Table 3.

TABLE 3
Comparison of Boc-aromatic amino acid-Gly-INH and Boc-aromatic amino
acid-Lys-INH, and DBDY-(Gly-INH)₂ and DBDY-(Lys-INH)₂ with respect to
hydrolysis by chymotrypsin, trypsin, papain, pepsin and thermolysin

| Substrate ^{b} | Chymotrypsin | Trypsin ^{c} | Papain ^{c} | Pepsin ^{c} | Thermolysin ^{c} |
|---|---|---|---|---|---|
| G1 | 38.7 ± 2.9 ^{d} | n.d. ^{f} | 62.8 ± 2.5 | 2.34 ± 0.45 | 0.74 ± 0.02 |
| G2 | 28.6 ± 3.4 ^{d} | n.d. | 51.0 ± 3.5 | 1.61 ± 0.05 | n.d. |
| G3 | 17.3 ± 5.5 ^{d} | n.d. | 58.8 ± 6.2 | 3.21 ± 0.45 | 0.74 ± 0.05 |
| G4 | n.d. | n.d. | 62.2 ± 2.1 | 2.98 ± 0.07 | 0.74 ± 0.03 |
| L1 | 37.6 ± 5.2 ^{e} | 69.6 ± 4.1 | 77.4 ± 5.8 | 0.74 ± 0.04 | n.d. |
| L2 | 53.2 ± 4.8 ^{e} | 81.2 ± 4.9 | 89.1 ± 2.5 | n.d. | n.d. |
| L3 | 58.3 ± 1.2 ^{e} | 85.1 ± 3.7 | 93.1 ± 8.9 | n.d. | 4.04 ± 0.08 |
| L4 | 7.15 ± 0.47 ^{c} | 46.8 ± 1.7 | 44.9 ± 2.8 | 1.04 ± 0.03 | n.d. |

^{a} Whereas the initial concentration of Boc-aromatic amino acid-Gly-INH and Boc-aromatic amino acid-Lys-INH was 0.2 mM, the initial concentration of DBDY-(Gly-INH)₂ and DBDY-(Lys-INH)₂ was 0.1 mM. A protease was added at a concentration of 10 μM. The values in the table represent the degree of hydrolysis (%) after reacting for 2 hours. Experiments were performed at least 3 times. The average and standard deviation thereof were shown.
^{b} Reference numbers in the substrate column denoted the following: G1 = Boc-Tyr- Gly-INH; G2 = Boc-Phe-Gly-INH; G3 = Boc-Trp-Gly-INH; G4 = DBDY-(Gly-INH)₂; L1 = Boc-Tyr-Lys-INH; L2 = Boc-Phe-Lys-INH; L3 = Boc-Trp-Lys-INH; and L4 = DBDY-(Lys-INH)₂.
^{c} Upon hydrolysis, INH was detected as a hydrolysate.
^{d} Gly-INH was detected as a hydrolysate.
^{e} Lys-INH was detected as a hydrolysate.
^{f} n.d. = no hydrolysis was detected.

The effect of DY was clearly shown only in the reaction with chymotrypsin which has been known to have specificity for a hydrophobic amino acid such as an aromatic amino acid at a P₁ position. The experimented Boc-aromatic amino acid-Gly-INH and Boc-aromatic amino acid-Lys-INH both were hydrolyzed by chymotrypsin (refer to Table 3), and the hydrolysates were Gly-INH and Lys-INH (not shown). However, DBDY-(Gly-INH)₂ and DBDY-(Lys-INH)₂ were hardly hydrolyzed by chymotrypsin. Although dityrosine is hydrophobic, it is considered not bulky enough to match the active site of chymotrypsin.

### Experimental Example 4

### Selectivity of DBDY-(Gly-INH)₂ and DBDY-(Lys-INH)₂ for Cathepsins B, L and S

Since cathepsins B, L and S belong to cysteine proteases, DBDY-(Gly-INH)₂ and DBDY-(Lys-INH)₂ were experimented for the selective hydrolysis by these cathepsins. The results of experiments were illustrated in FIG. 20. Since the hydrolysis by cathepsin B was faster than the hydrolyses by papain and chymopapain, the reaction was performed with a substrate (concentration: 1.0 μM) and a protease (concentration: 10 mM) of which the concentrations were set lower than those in Experimental Example 2 (substrate (concentration: 2.5 μM) and protease (concentration: 0.1 μM) reacted for two hours) for 30 minutes in FIG. 20. C1, C2, CB, CL, and CS represented on an X-axis respectively denote papain, chymopapain, cathepsin B, cathepsin L, and cathepsin S. Each experiment was repeated at least three times.

As expected, DBDY-(Lys-INH)₂ was hydrolyzed by cathepsins B and L. However, interestingly, DBDY-(Gly-INH)₂ was hydrolyzed only by cathepsin B. Cathepsin S has been known to prefer an aliphatic amino acid rather than an aromatic amino acid at a P₂ position. Accordingly, DBDY-(Lys-INH)₂ and DBDY-(Gly-INH)₂ were not hydrolyzed by cathepsin S.

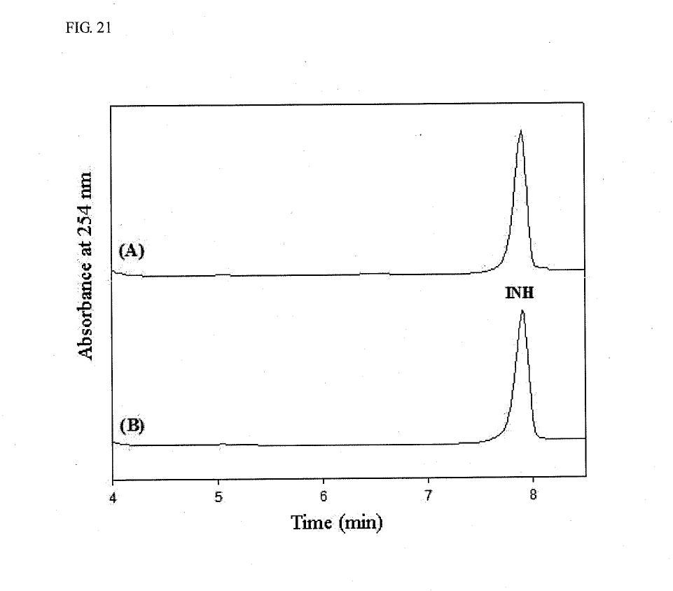

A HPLC analysis was performed to identify hydrolysates, and the result of the reaction with cathepsin B was illustrated in FIG. 21. The initial concentrations of each substrate and cathepsin B were respectively 0.1 mM and 0.1 μM. The reaction was performed for 5 hours.

In both results of the HPLC chromatogram of hydrolysates, peaks for INH were found at a retention time (RT) of 8 minutes, but peaks for Gly-INH (RT=4.9 min) and Lys-INH (RT=7.1 min) were not found. Cathepsin B recognized glycine and lysine as an amino acid at a P₁ position when cathepsin B hydrolyzed DBDY-(Gly-INH)₂ and DBDY-(Lys-INH)₂. Cathepsin L also recognized lysine as an amino acid at a P₁ position when cathepsin L hydrolyzed DBDY-(Lys-INH)₂ (HPLC chromatogram for cathepsin L was not shown).

The hydrolysis of DBDY-(Gly-INH)₂ and DBDY-(Lys-INH)₂ by cysteine proteases was determined to depend on Michaelis-Menten kinetics. Table 4 is a summary of the kinetics parameters.

TABLE 4
Kinetics parameters with respect to hydrolysis of DBDY-
(Gly-INH)₂ and DBDY-(Lys-INH)₂ by cysteine protease

| Sub- | | | | |
|---|---|---|---|---|
| strate ^{a} | Protease | K_{M}(μM)^{b} | K_{cat}(min⁻¹)^{b} | K_{cat}/K_{M}(M⁻¹s⁻¹) |
| G4 | Papain | 7.72 ± 1.25 | 1.54 ± 0.30 | 3.32 × 10³ |
| | Chymopapain | 4.00 ± 0.25 | 0.34 ± 0.03 | 1.43 × 10³ |
| | Cathepsin B | 2.88 ± 0.15 | 0.67 ± 0.06 | 3.87 × 10³ |
| | Cathepsin L | 12.9 ± 3.69 | 0.09 ± 0.02 | 1.13 × 10² |
| | Cathepsin S | n.d. ^{c} | | |
| L4 | Papain | 7.20 ± 0.80 | 1.04 ± 0.11 | 2.41 × 10³ |
| | Chymopapain | 2.20 ± 0.28 | 0.03 ± 0.01 | 1.90 × 10² |
| | Cathepsin B | 2.01 ± 0.05 | 2.17 ± 0.15 | 1.80 × 10⁴ |
| | Cathepsin L | 1.17 ± 0.09 | 0.20 ± 0.01 | 2.89 × 10³ |

| | |
|---|---|
| Cathepsin S | n.d. |

^{a} Reference numbers in the substrate column denoted the following: G4 = DBDY-(Gly-INH)₂; and L4 = DBDY-(Lys-INH)₂.
^{b}Experiments were performed at least 3 times, and the average and standard deviation thereof were shown.
^{c} n.d. = no hydrolysis was detected.

The K_{M} values were shown in the range of 1.17 to 12.9 μM, and were relatively lower than those of the analytes of other proteases reported (4.6 to 404 μM). Particularly, the K_{M} value of cathepsin B with respect to DBDY-(Gly-INH)₂ was 2.88 μM, and was comparable with the K_{M} values of cathepsin B with respect to other substances. That is, the K_{M} value of cathepsin B with respect to Abz-Phe-Phe-Dap(Dnp)-Trp-OH was 2.1 μM, the K_{M} value of cathepsin B with respect to Abz-Phe-Arg-Dap(Dnp)-Trp-OH was 3.0 μM, the K_{M} value of cathepsin B with respect to Abz-Gly-Ile-Val-Arg-Ala-Lys(Dnp)-OH was 5.9 Abz and Dap(Dnp) respectively refer to o-aminobenzoyl and 3-(2,4-dinitrophenyl)-2,3-diaminopropionic acid.

DBDY-(Gly-INH)₂ is advantageous in the selective and sensitive analysis of cathepsin B in that there is no need for expensive and complicated equipment for an automatic peptide synthesis.

In conclusion, as a result of the experiment in which a diaromatic amino acid bonded with a protease-preferred amino acid and two molecules of isoniazid was tested whether it may be used as a substrate for the analysis of proteases, the fluorescence of DBDY (λₑₓ=284 to 320 nm; λₑₘ=400 to 420 nm) was removed in DBDY-(amino acid-INH)₂ due to the removal effect of INH, but was restored when DBDY-(amino acid-INH)₂ was catalyzed and hydrolyzed by proteases inducing the emission of amino acid-INH or INH. Particularly, cathepsins B and L were sensitively and selectively analyzed by DBDY-(Lys-INH)₂ and DBDY-(Gly-INH)₂.

According to an embodiment of the present invention, the sensitive and accurate fluorescence detection of a cathepsin can be realized using a diaromatic amino acid bonded with a cathepsin-preferred amino acid and isoniazid which is a quencher, and this diaromatic amino acid-based substrate has an advantage of a simple structure and a low manufacturing cost.

Further, the fluorescent substrate can serve to diagnose various types of cathepsin-overexpressing cancers, or to screen for cathepsin inhibitors.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. A compound of Formula 1: 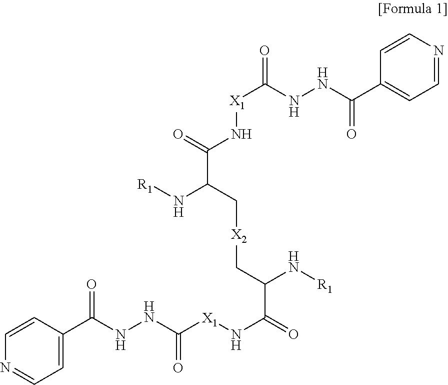
wherein X₁ represents glycine, lysine, arginine or leucine,
X₂
represents 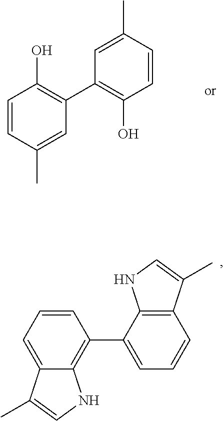
and
R₁ represents an amine-protecting group.

2. The compound of claim 1, wherein X₁ represents glycine or lysine,
X₂ represents 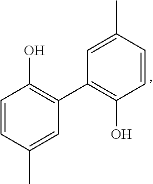 [SMILES: Cc1ccc(O)c(-c2cc(C)ccc2O)c1]
R₁ is a tert-butyloxycarbonyl group, a 9-fluorenylmethyloxycarbonyl group, a benzyloxycarbonyl group, polyethylene glycol, or NH₂CHR′ₙ, where R′ is an amino acid moiety, and n represents an integer in a range of 1 to 20.

3. A method of preparing a compound of Formula 1, comprising:
preparing a compound of Formula 4 by reacting a compound of Formula 2 and a compound of Formula 3;
removing an amine-protecting group R₁ from the compound of Formula 4; and
preparing the compound of Formula 1 by reacting a compound of Formula 4 from which the amine-protecting group R₁ is removed with a compound of Formula 5: 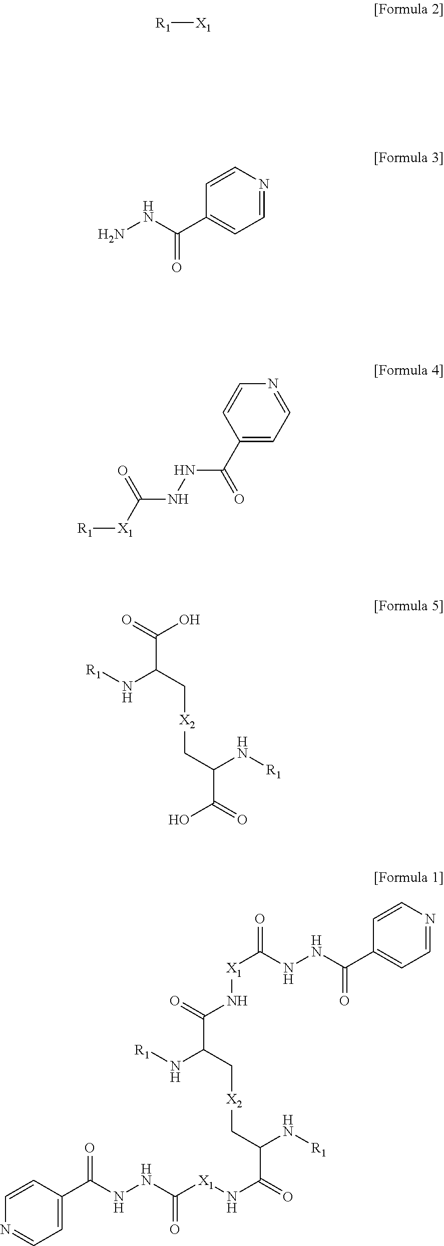
wherein, X₁ represents glycine, lysine, arginine or leucine,
X₂
represents 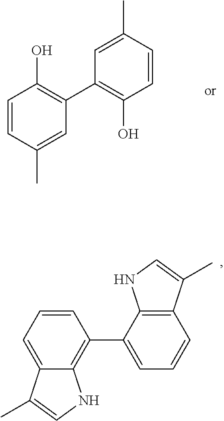
R₁ represents an amine-protecting group.

4. The method of claim 3, wherein a reaction to prepare the compound of Formula 4 or the compound of Formula 1 is performed in the presence of a catalyst and a crosslinking agent.

5. The method of claim 4, wherein the catalyst is at least one of a carbodiimide-based compound and NHS or sulfo-NHS; carbodiimidazole; or Woodward's reagent K.

6. The method of claim 4, wherein the crosslinking agent is one or more types selected from the group consisting of dithiobis(succinimidylpropionate) (DSP), 3,3′-dithiobis(sulfosuccinimidylpropionate) (DTSSP), disuccinimidyl suberate (DSS), bis(sulfosuccinimidyl)suberate (BS3), ethylene glycolbis(succinimidylsuccinate) (EGS), N,N′-disuccinimidyl carbonate (DSC), N-succinimidyl 3-(2-pyridyldithio)propionate (SPDP), succinimidyloxycarbonyl-α-(2-pyridyldithio)toluene (SMPT) and succinimidyl 4-(N-maleimidomethyl)-cyclohexane-1-carboxylate (SMCC).

7. The method of claim 3, wherein 1 to 3 moles of a compound of Formula 3 are added per mole of a compound of Formula 2.

8. The method of claim 3, wherein a reaction to prepare a compound of Formula 4 or a compound of Formula 1 is performed in the presence of one or more types of an organic solvent selected from the group consisting of dichloromethane, dimethylformamide, 1,4-dioxane, and methylene chloride.

9. The method of claim 3, wherein 2 to 5 moles of a compound of Formula 4 are added per mole of a compound of Formula 5.

10. A cathepsin-detecting sensor, comprising a compound of Formula 1: 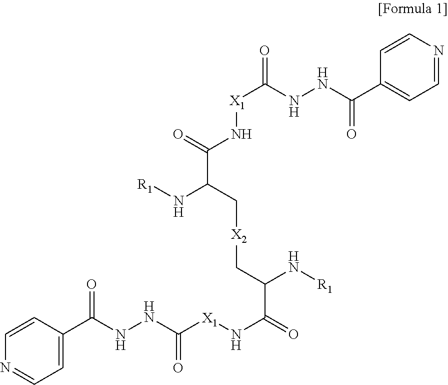
wherein X₁ represents glycine, lysine, arginine or leucine,
X₂ represents 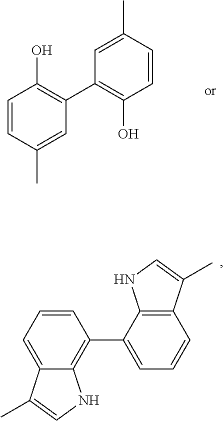
and
R₁ represents an amine-protecting group.

11. The sensor of claim 10, wherein, X₁ represents glycine or lysine,
X₂ represents 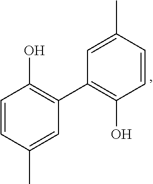 [SMILES: Cc1ccc(O)c(-c2cc(C)ccc2O)c1]
R₁ is a tert-butyloxycarbonyl group, a 9-fluorenylmethyloxycarbonyl group, a benzyloxycarbonyl group, polyethylene glycol, or NH₂CHR′ₙ, where R′ is an amino acid moiety, and n represents an integer in a range of 1 to 20.

12. The sensor of claim 10, wherein the cathepsin is cathepsin B or cathepsin L.

13. A cathepsin-detecting composition, comprising a compound of Formula 1: 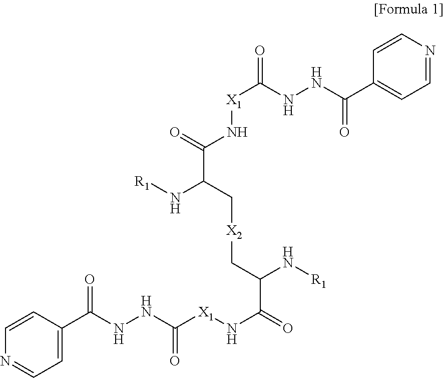
wherein X₁ represents glycine, lysine, arginine or leucine,
X₂
represents 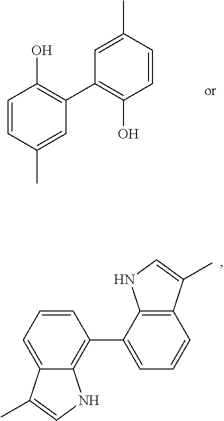
and
R₁ represents an amine-protecting group.

14. The composition of claim 13, wherein X₁ represents glycine or lysine,
X₂ represents 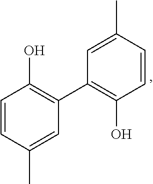 [SMILES: Cc1ccc(O)c(-c2cc(C)ccc2O)c1]
R₁ is a tert-butyloxycarbonyl group, a 9-fluorenylmethyloxycarbonyl group, a benzyloxycarbonyl group, polyethylene glycol, or NH₂CHR′ₙ, where R′ is an amino acid moiety, and n represents an integer in a range of 1 to 20.

15. The composition of claim 13, further comprising a phosphate buffer solution including NaCl and ethylenediaminetetraacetic acid (EDTA) and having a pH in a range of 3 to 10.

16. A diagnostic kit for a cathepsin-overexpressing disease, comprising a compound of Formula 1: 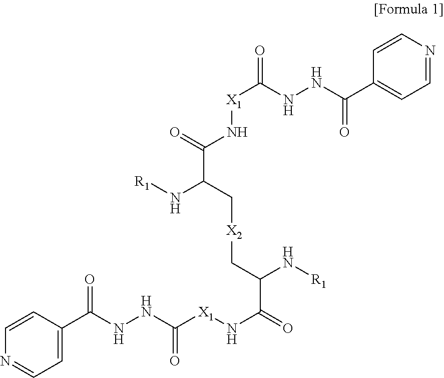
wherein X₁ represents glycine, lysine, arginine or leucine,
X₂
represents 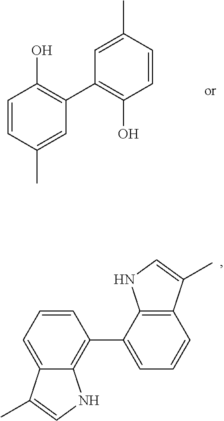
and
R₁ represents an amine-protecting group.

17. The diagnostic kit of claim 16, wherein X₁ represents glycine or lysine,
X₂ represent 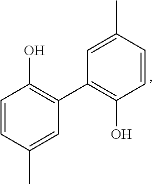 [SMILES: Cc1ccc(O)c(-c2cc(C)ccc2O)c1]
R₁ is a tert-butyloxycarbonyl group, a 9-fluorenylmethyloxycarbonyl group, a benzyloxycarbonyl group, polyethylene glycol, or NH₂CHR′ₙ, where R′ is an amino acid moiety, and n represents an integer in a range of 1 to 20.

18. The diagnostic kit of claim 16, wherein the disease is cancer.

19. A composition for screening for a cathepsin inhibitor, comprising a compound of Formula 1: 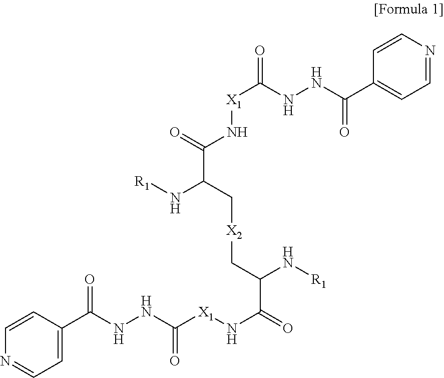
wherein X₁ represents glycine, lysine, arginine or leucine,
X₂
represents 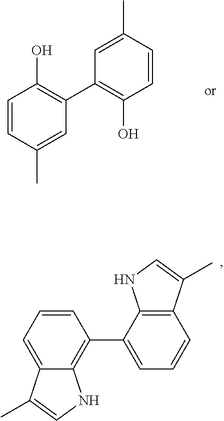
and
R₁ represents an amine-protecting group.

20. The composition of claim 19, wherein X₁ represents glycine or lysine,
X₂ represents 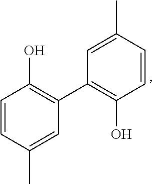 [SMILES: Cc1ccc(O)c(-c2cc(C)ccc2O)c1]
R₁ is a tert-butyloxycarbonyl group, a 9-fluorenylmethyloxycarbonyl group, a benzyloxycarbonyl group, polyethylene glycol, or NH₂CHR′ₙ, where R′ is an amino acid moiety, and n represents an integer in a range of 1 to 20.
